(19) United States
(12) Patent Application Publication
(10) Pub. No.: US 2024/0287579 A1
(43) Pub. Date: Aug. 29, 2024

(54) METHOD FOR ANALYZING DNA METHYLATION USING NEXT GENERATION SEQUENCER AND METHOD FOR CONCENTRATING SPECIFIC DNA FRAGMENTS

(71) Applicant: Naomi YAMAKAWA, Kawagoe-shi (JP)

(72) Inventor: Naomi YAMAKAWA, Kawagoe-shi (JP)

(21) Appl. No.: 18/432,060

(22) Filed: Feb. 5, 2024

Related U.S. Application Data
(62) Division of application No. 16/759,995, filed on Sep. 15, 2020, now abandoned.

(30) Foreign Application Priority Data
Oct. 30, 2017 (JP) 2017-209534

(51) Int. Cl. C12Q 1/6806

(52) U.S. Cl. CPC C12Q 1/6806; C12Q 2521/331; C12Q 2521/501; C12Q 2535/122

(57) ABSTRACT

This invention provides technology of DNA methylation analysis including: (1) a step of digesting DNA to be analyzed with a restriction enzyme(s) containing methylated cytosine or possibly methylated cytosine in a recognition sequence(s), wherein the recognition site is affected by the methylation; (2) a step of treating the mixture of DNA fragments obtained in the step (1) with ligase to ligate them; (3) a step of determining the base sequence of each DNA constructs included in the mixture of DNA constructs obtained in the step (2); and (4) a step of comparing the base sequence information of each recognition sites and its surrounding sequences, obtained in the step (3), to a known genome sequence; determining whether said each recognition site is not cleaved with said restriction enzyme or cleaved with said restriction enzyme then regenerated by ligation with said ligase; and finally, determining each methylation states of each recognition sites.

### CROSS-REFERENCE TO RELATED APPLICATIONS

This Application is a divisional application claiming the benefit of priority of the co-pending U.S. Utility Non-Provisional Patent application Ser. No. 16/759,995, with a filing date of Sep. 15, 2020, which claims the benefit of priority of International Patent Application No. PCT/JP2018/040251, with an international filing date of Oct. 30, 2018, which designated the United States, which claims the benefit of priority of Japan Patent Application No. 2017-209534, with a filing date of Oct. 30, 2017, the entire disclosures of all Applications are expressly incorporated by reference in their entirety herein.

### PRIOR ART

Patent Literature Reference No. 1: International Publication No. 2009/131223
Non-patent Literature No. 1: Lister R, O'Malley R C, Toction-Filipini J, Gregory B D, Berry C C, Millar A H and Ecker J R., High Integrated Single-base resolution maps of the epigenome in Arabidopsis., Cell, 2008; 133:523-536.
Non-patent Literature No. 2: Hatada I, et al., Genome-wide profiling of promoter methylation in human., Oncogene, 2006; 25:3059-3064.
Non-patent Literature No. 3: Ushijima T, Morimura K, Hosoya Y, Okonogi H, Tatematsu M, Sugimura T, Nagao M., Establishment of methylation-sensitive-differential differential and IS and polypho- and hypermethylated genomic fragments in mouse liver tumours., Proc Natl Acad Sci USA. 1997 Mar. 18; 94(6):2284-9.

### BACKGROUND OF THE INVENTION

The present invention relates to methods of DNA methylation analysis.

Conventional DNA methylation assays are roughly divided into methods using methylation-sensitive restriction enzymes, methods using bisulfite, and methods using affinity columns.

In the bisulfite method, the next generation sequencer (NGS) is widely used for genome-wide methylation analysis, and this method is becoming a main stream (Non-patent Document 1). As for the bisulfite method, a large number of unmethylated cytosines in the genome are converted to uracil by bisulfite treatment, and these uracils are further converted to thymine by subsequent polymerase chain reaction (PCR) amplification reaction. These processes increase the thymine content of particular analyte DNA fragments, decrease the sequence complexity of the DNA fragments, and thus produce unmapped sequence information onto the reference genome sequence in the mapping process. Therefore, depending on the equipment used and the length of the DNA strand obtained, approximately one-third of the obtained fragment information might be discarded as unmappable DNA sequence information. Because of the reason, the analysis cost is relatively high and it is not fully utilized in life science research.

The several methods of quantitative methylation analysis of cytosines in the -CpG- sequence are used to analyze cytosine in the restriction enzyme recognition site quantitatively by using a combination of cytosine-methylation-sensitive and non-sensitive restriction enzymes; MIAMI (Microarray-based Integrated Analysis of Methylation by Isoschizomers), Non-Patent Document 2; MS-RDA (Methylation-Sensitive Representational Difference Analysis), Non-Patent Document 3. For example, methylation analysis of cytosine in the restriction enzyme recognition site can be performed quantitatively by using a combination of methylation-sensitive Hpa II and methylation-insensitive MspI restriction enzymes.

In general, the methylation rate is usually quantitated for a particular site discontinuously from 0% to 100% in the methylation rate, so good reproducibly can be performed as long as a highly quantitative method is used for the analysis.

However, for the analyses, a combination of two restriction enzymes with the same recognition sequence but with different sensitivity against cytosine-methylation sequence are required to evaluate whether or not the cytosine in the recognition sequence is methylated. Since such as the combination of restriction enzymes is rare, the assayable region is very limited within their recognition sites. This systematic issue eliminates some genes from its methylation analysis; there is a great limitation on the flexibility of setting the region to be analyzed. This has been a major obstacle in this research field.

In addition, because the sequence at which cytosine can be methylated in the plant genome is -CpNpG-, it is not an exaggeration to say that there is no combination of restriction enzymes that meets the above conditions, and that the progress in plant epigenomic analysis is much slower than that in epigenomic analysis for animals.

One conventional method to analyze the cytosine methylation is to completely digests the analyte genomic DNA with methylation-insensitive restriction enzymes, conjugate adapters that specifically attach to their sticky ends, digest the conjugate with methylation-sensitive restriction enzymes, and then analyze whether or not the conjugated adapter can be removed with digestion of methylation-sensitive restriction enzymes (Patent Document 1). In this method, the adapters which can specifically conjugate to the restriction site produced with the used enzymes must individually be designed, and thus the reaction requires this complicated, multistep process. In addition, this method and other DNA methylation analysis requires the DNA array to be prepared for a specific DNA array corresponding to the methylation assessment region. And also, the analyte DNA is subjected to fluorescent labeling, hybridization with the DNA array for a long period of time, along with other steps which makes the procedure extremely complicated.

Furthermore, it is impossible to identify which end of the DNA fragment is methylated because of DNA array analysis. Therefore, only methylation typing for each DNA fragment, rather than each recognition site, can be performed.

### SUMMARY OF THE INVENTION

As we described above, there are various limitations and weak points in conventional methods, and this has been a major obstacle for DNA methylation analysis to be widely applied. The challenge of the present invention is to provide a new method of methylation analysis capable of overcoming these limitations and drawbacks, and to also provide a method to obtain a particular group of methylated DNA fragments capable of improving the analytical efficiency.

This invention relates to:
1. A method for determining a methylation state in a DNA to be analyzed, with said method comprising of:
   (1) digesting the DNA to be analyzed with a restriction enzyme whose recognition site includes methylated cytosine or cytosine to be methylated and is affected by methylation;
   (2) ligating a mixture of DNA fragments obtained in step (1) with a ligase;
   (3) determining a nucleotide sequence of each DNA construct included in a mixture of DNA constructs obtained in step (2); and
   (4) comparing a nucleotide sequence of each recognition site of the restriction enzyme and its flanking nucleotide sequences included in each nucleotide sequence information obtained in step (3) with a known genomic sequence, and determining whether each recognition site is a recognition site that has not been digested with the restriction enzyme, or a recognition site that has been digested with the restriction enzyme and regenerated by the ligation with the ligase, to thereby determine the methylation state at each recognition site.
2. The method according to claim 1, wherein the mixture of DNA fragments obtained in step (1) is ligated with the ligase in the presence of an adapter capable of being ligated to both of its ends, in step (2).
3. The method according to claim 1 or 2, wherein a desired DNA fragment group is fractionated from the mixture of DNA fragments obtained in step (1) before the ligation with the ligase, in step (2).
4. The method according to any one of claims 1 to 3, wherein a DNA amplification is carried out using a DNA polymerase with strand displacement activity after the ligation with the ligase, in step (2).
5. The method according to any one of claims 1 to 4, wherein a nucleotide sequence between adjacent recognition sites of the restriction enzyme is mapped to a known genomic sequence, and a sequence outside at least one of the adjacent recognition sites is compared with the mapped reference sequence, to thereby determine whether the recognition site is a recognition site that has not been digested with the restriction enzyme, or a recognition site that has been digested with the restriction enzyme and regenerated by the ligation with the ligase, in step (4).
6. The method according to any one of claims 1 to 5, wherein a methylation rate at a specific recognition site is determined by calculating a ratio of the recognition site that has not been digested with the restriction enzyme to the recognition site that has been digested with the restriction enzyme and regenerated by the ligation with the ligase, in step (4).
7. A concatenated long-chain DNA that holds methylation information, obtained by fragmentating genomic DNA with a methylation-sensitive restriction enzyme, and carrying out a multiple ligation using a ligase in the presence or absence of an adapter capable of being ligated to both ends thereof.
8. The concatenated long-chain DNA that holds methylation information according to claim 7, wherein, after the fragmentation by the treatment with the restriction enzyme, a desired DNA fragment group is fractionated from the obtained mixture of DNA fragments.
9. A concatenated long-chain DNA amplification product that holds methylation information, obtained by carrying out amplification with a DNA polymerase with strand displacement activity using the concatenated long-chain DNA that holds methylation information according to claim 7 or 8 as a template.
10. A method for obtaining a DNA fragment group, comprising:
   (1) digesting a DNA to be analyzed with a methylation-sensitive restriction enzyme whose recognition site includes methylated cytosine or cytosine to be methylated and which generates a cohesive end;
   (2) ligating labeled adapters which do not generate a recognition site of the methylation-sensitive restriction enzyme, to both ends of the DNA fragments obtained in step (1);
   (3) digesting the labeled DNA constructs obtained in step (2) with a methylation-insensitive restriction enzyme which recognizes the same recognition site as the methylation-sensitive restriction enzyme and which generates a cohesive end; and
   (4) removing only the labeled DNA fragments from the mixture of DNA fragments obtained in step (3) using a binding partner specific to the label, to obtain a DNA fragment group consisting of DNA fragments in which methylated cytosines are included in both cohesive ends at both ends.
11. A method for obtaining a DNA fragment group, comprising:
   (1) digesting a DNA to be analyzed with a methylation-sensitive restriction enzyme whose recognition site includes methylated cytosine or cytosine to be methylated and which generates a cohesive end;
   (2) converting both ends of the DNA fragments obtained in step (1) into blunt ends in the presence of labeled deoxynucleoside triphosphate;
   (3) digesting the labeled DNA fragments obtained in step (2) with a methylation-insensitive restriction enzyme which recognizes the same recognition site as the methylation-sensitive restriction enzyme and which generates a cohesive end; and
   (4) removing only the labeled DNA fragments from the mixture of DNA fragments obtained in step (3) using a binding partner specific to the label, to obtain a DNA fragment group consisting of DNA fragments in which methylated cytosines are included in both cohesive ends at both ends.
12. A concatenated long-chain DNA that holds methylation information, obtained by multiple-ligating the DNA fragment group consisting of DNA fragments in which methylated cytosines are included in both cohesive ends at both ends, using a ligase, said DNA fragment group being obtained by the method according to claim 10 or 11.
13. A concatenated long-chain DNA amplification product that holds methylation information, obtained by carrying out amplification, using the concatenated long-chain DNA that holds methylation information according to claim 12 as a template.
14. A method for obtaining a DNA fragment group, comprising:
   (1) digesting a DNA to be analyzed with a methylation-sensitive restriction enzyme whose recognition site includes methylated cytosine or cytosine to be methylated and which generates a cohesive end;
   (2) ligating stem-loop adapters which do not generate a recognition site of the methylation-sensitive restriction enzyme, to both ends of the DNA fragments obtained in step (1);
   (3) digesting the DNA constructs obtained in step (2) with a methylation-insensitive restriction enzyme which recognizes the same recognition site as the methylation-sensitive restriction enzyme and which generates a cohesive end;
   (4) ligating nuclease-resistant labeled adapters whose 5′-end shows nuclease resistance and which generate the recognition site of the methylation-insensitive restriction enzyme, to each cohesive end of the DNA fragments obtained in step (3); and
   (5) treating the DNA constructs obtained in step (4) with a single-strand-specific endonuclease followed by a combination of double-strand-specific and single-strand-specific endonucleases, to completely digest only DNA fragments to which the stem-loop adapters are ligated, and to obtain a DNA fragment group consisting of DNA fragments to which the nuclease-resistant labeled adapters are ligated at both ends thereof.
15. A method for obtaining a DNA fragment group, comprising:
   (1) digesting the DNA fragment group obtained by the method according to claim 14 with the methylation-insensitive restriction enzyme described in claim 14; and
   (2) removing the nuclease-resistant labeled adapters from the digested product obtained in step (1) using a binding partner specific to the label, to obtain a DNA fragment group consisting of DNA fragments in which methylated cytosines are included in both cohesive ends at both ends.
16. A concatenated long-chain DNA that holds methylation information, obtained by multiple-ligating the DNA fragment group consisting of DNA fragments in which methylated cytosines are included in both cohesive ends at both ends, using a ligase, said DNA fragment group being obtained by the method according to claim 15.
17. A concatenated long-chain DNA amplification product that holds methylation information, obtained by carrying out amplification, using the concatenated long-chain DNA that holds methylation information according to claim 16 as a template.
18. A method for a DNA fragment group, comprising:
   (1) digesting a DNA to be analyzed with a methylation-sensitive restriction enzyme whose recognition site includes methylated cytosine or cytosine to be methylated and which generates a cohesive end;
   (2) ligating nuclease-resistant labeled adapters whose 5′-end shows nuclease resistance and which have a restriction enzyme recognition site consisting of 8 nucleotides or more and do not generate a recognition site of the methylation-sensitive restriction enzyme, to both ends of the DNA fragments obtained in step (1);
   (3) digesting the DNA constructs obtained in step (2) with a methylation-insensitive restriction enzyme which recognizes the same recognition site as the methylation-sensitive restriction enzyme and which generates a cohesive end;
   (4) ligating stem-loop adapters to both ends of the DNA fragments obtained in step (3);
   (5) treating the DNA constructs obtained in step (4) with a single-strand-specific endonuclease followed by a combination of double-strand-specific and single-strand-specific endonucleases, to completely digest only DNA fragments to which the stem-loop adapters are ligated, and to obtain a DNA fragment group consisting of DNA fragments to which the nuclease-resistant labeled adapters are ligated at both ends thereof.
19. A method for a DNA fragment group, comprising:
   (1) digesting the DNA fragment group obtained by the method according to claim 18, with a restriction enzyme which recognizes the restriction enzyme recognition site consisting of 8 nucleotides or more in the labeled adapter described in claim 18; and
   (2) removing the nuclease-resistant labeled adapters from the digested product obtained in step (1) using a binding partner specific to the label, to obtain a DNA fragment group consisting of DNA fragments in which cytosines are included in both cohesive ends at both ends.
20. A concatenated long-chain DNA that holds methylation information, obtained by multiple-ligating the DNA fragment group consisting of DNA fragments in which methylated cytosines are included in both cohesive ends at both ends, using a ligase, said DNA fragment group being obtained by the method according to claim 19.
21. A concatenated long-chain DNA amplification product that holds methylation information, obtained by carrying out amplification, using the concatenated long-chain DNA that holds methylation information according to claim 20 as a template.
22. The method for a DNA fragment group according to any one of claims 10, 11, 14, 15, 18, and 19, wherein, after at least one digestion step of the restriction enzyme digestion steps or the nuclease digestion steps, a desired DNA fragment group is fractionated from the obtained mixture of DNA fragments.
23. A method for determining a methylation state in a DNA to be analyzed, comprising of; determining a nucleotide sequence of the concatenated long-chain DNA that holds methylation information according to any one of claims 7, 8, 12, 16, and 20, or the concatenated long-chain DNA amplification product that holds methylation information according to any one of claims 9, 13, 17, and 21.

In the analytical technique developed here, multiple DNA methylation-sensitive restriction enzymes can be freely used in combination, and no specific adapter designing is required for each restriction enzyme to be used. Since the target of the methylation analysis is the restriction enzyme recognition site itself, the area to be analyzed can be further extended, thereby achieving an extremely high resolution on the analysis when compared to methods using the aforementioned DNA arrays.

Another major feature of the invention is the ability to combine multiple methylation-sensitive restriction enzymes in the assay. This allows us to increase the number of the restriction enzyme recognition sites to be analyzed at the same time, and dramatically improve the resolution of analysis compared to the conventional analytical methods using restriction enzymes. Many applications of this method are expected in the medical field, such as diagnosis to judge whether cells are benign or malignant cancer cells, and analysis to predict the original tissue of unknown primary tumors to plan an appropriate treatment strategy. The human body is composed of approximately 200 cell types; therefore, referring a particular cell to the characteristic methylation patterns of different cell types, which are extracted from genomic DNA methylation analyses performed in advance, can make us predict the original cell species of analyte cancer cells. The present technology has been developed in view of such medical applications, so that it has provided us sufficient analytical resolution, at lower assay cost, and while using automation.

Furthermore, the methylation analysis method of the present invention does not require to use two restriction enzymes that recognize the same sequence, and thus the methylation of cytosine in the -CpNpG- sequence found in plants can also be analyzed.

And also, in conventional DNA amplification (e.g., PCR), all methylated cytosines are substituted with unmethylated cytosine (i.e., methylated information disappears) in the resulting DNA amplification products; however, in the present invention, even after when the DNA amplification is performed, the methylation state before digestion can be determined as in described below.

### BRIEF DESCRIPTION OF DRAWINGS

FIG. 1 is an explanatory diagram showing the structure of genomic DNA (partial regions) before digestion with methylation-sensitive restriction enzyme(s) to understand the structure of the concatenated long-chain DNA that is important in the methods of the present invention.

FIG. 2 is an explanatory diagram showing the structure of the concatenated long-chain DNA obtained by ligation performed after digestion of the genomic DNA shown in FIG. 1 with the methylation-sensitive restriction enzymes of HpaII and HhaI.

FIG. 3 is a photograph showing results of electrophoresis of a DNA fragment mixture obtained by digesting the genomic DNA of human fibrosarcoma cell line HT-1080 with the methylation-sensitive restriction enzymes HpaII and HhaI (lane 1), and a concatenated long-chain DNA (lane 2) obtained by ligation of said DNA fragment mixture.

### DETAILED DESCRIPTION OF THE INVENTION

As used herein, the term “methylation of cytosine” refers to the general methylation modification of cytosine with respect to cellular differentiation and biocontrol, and also includes hydroxymethylation for example, in addition to the methylation of cytosine.

### Methylation Analysis Method:

A method for determining the state of methylation of the DNA to be analyzed of the present invention (hereinafter referred to as the methylation analysis method of the present invention) comprises of:
(1) digesting the DNA to be analyzed with a restriction enzyme whose recognition site includes methylated cytosine or cytosine to be methylated and is affected by methylation (digestion step);
(2) ligating a mixture of DNA fragments obtained in step (1) with a ligase (ligation step);
(3) determining a nucleotide sequence of each DNA construct (concatenated long-chain DNA) included in a mixture of DNA constructs obtained in step (2) (sequencing step); and
(4) comparing a nucleotide sequence of each recognition site of the restriction enzyme and its flanking nucleotide sequences included in each nucleotide sequence information obtained in step (3) with a known genomic sequence, and determining whether each recognition site is a recognition site that has not been digested with the restriction enzyme, or a recognition site that has been digested with the restriction enzyme and regenerated by the ligation with the ligase, to thereby determine the methylation state at each recognition site (analyzing step).

In the method of methylation analysis of the present invention, instead of said step (2), can be carried out by:
(2′) treating a mixture of DNA fragments obtained in said step (1) with ligase to concatenate, and then performing DNA amplification with a strand displacement DNA polymerase after said ligase treatment (concatenation and amplification processes).

In the method of methylation analysis of the present invention, ligase treatment in step (2) or step (2′) may be performed in the presence of adapter(s) capable of being ligated to both ends of the DNA fragments in the mixture obtained in step (1).

In step (1) of the method of methylation analysis of the present invention, that is, in the digestion step, a sample DNA to be analyzed is digested with restriction enzyme(s).

The DNA to which the methylated analytical method of the present invention can be applied is not particularly limited as long as it is a DNA that may contain methylated cytosine or potentially methylated cytosine, and may include, for example, genomic DNA of a cell (e.g., animal cells or plant cells) or a mixture of free DNA fragments present in biological samples or a sample derived therefrom (e.g., blood, plasma, serum, urine, lymph fluid, cerebrospinal fluid, saliva, ascites, amniotic fluid, mucus, milk, bile, gastric fluid, or artificial dialysis fluid after dialysis, etc.), and artificially synthesized DNAs.

The digestive enzymes used in step (1) include methylated cytosine or potentially methylated cytosine in the recognition sequence and are not particularly limited as long as the restriction enzyme can be affected by methylation at the recognition site, for example, methylation-sensitive restriction enzymes, methylation-dependent restriction enzymes, and preferably methylation-sensitive restriction enzymes. It is also preferable that the recognition sequence showed to be protruding end when the DNA is cleaved with the restriction enzyme. Furthermore, when two or more restriction enzymes are used, a combination of restriction enzymes which both produce the same sequence at the protruding ends can be used. However, the combination of restriction enzymes is not particularly restricted as long as it is possible to polymerize the DNA fragments obtained by the digestion with multiple restriction enzymes to form a long-chain or to form a template for DNA amplification by partially circularizing the DNA fragments. The methylation-sensitive restriction enzymes that can be used in the present invention are illustrated in Table 1.

TABLE 1

| Restriction | Recognition |
|---|---|
| Enzyme | Sequence |
| Aat II | GACGT↓C |
| Acc II | CG↓CG |
| Aor 13H I | T↓CCGGA |
| Aor 51H I | AGC↓GCT |
| BspT104 I | TT↓CGAA |
| BssH II | G↓CGCGC |
| BstUI | CG↓CG |
| Cfr 10 I | R↓CCGGY |
| Cla I | AT↓CGAT |
| Cpo I | CG↓GWCCG |
| Eco 52 I | C↓GGCCG |
| Hae II | RGCGC↓Y |
| HinPII | G↓CGC |
| Hpa II | C↓CGG |
| HpyCH4IV | A↓CGT |
| Hha I | GCG↓C |
| Mlu I | A↓CGCGT |
| Nae I | GCC↓GGC |
| NgoM IV | G↓CCGGC |
| Not I | GC↓GGCCGC |
| Nru I | TCG↓CGA |
| Nsb I | TGC↓GCA |
| PmaC I | CAC↓GTG |
| Psp 1406 I | AA↓CGTT |
| Pvu I | CGAT↓CG |
| Sac II | CCGC↓GG |
| Sal I | G↓TCGAC |
| Sma I | CCC↓GGG |
| SnaB I | TAC↓GTA |
| Xho I | C↓TCGAG |

The sample DNA to be analyzed can be digested with methylation-sensitive restriction enzymes, for example when a particular cytosine involved in methylation sensitivity in a recognition site (hereinafter referred to as unmethylated recognition site) is not chemically modified by processes such as methylation. On the other hand, the DNA cleavage is strongly inhibited at the recognition site when a particular cytosine is methylated (also including hydroxymethylated; hereinafter referred to as the methylated recognition site).

Accordingly, a DNA fragment mixture obtained after adequate digestion with methylation-sensitive restriction enzymes is going to be the mixture of DNA fragments whose ends are protruding end(s) and/or blunt end(s) derived from unmethylated recognition sites; and thus the methylated recognition site and their surrounding (upstream and downstream) sequence of each DNA fragment are kept intact.

On the other hand, digestion of the DNA to be analyzed with a methylation-dependent restriction enzyme (e.g., McrBC) results in DNA cleavage at the recognition site (i.e., methylated recognition site) at which point a particular cytosine responsible for methylation dependence in the recognition site is methylated, while the cleaving of the DNA is strongly inhibited at the recognition site (i.e., unmethylated recognition site) at which point said particular cytosine is not methylated.

Accordingly, a mixture of DNA fragments obtained after adequate digestion with methylation-dependent restriction enzymes is to be the DNA fragments mixture whose ends are protruding or blunt ends derived from the methylation recognition site, and then the unmethylated recognition site and its surrounding (upstream and downstream) sequences are thus kept intact in the sequence of each DNA fragment.

Hereinafter, steps (2) to (4) in the methylation analysis method of the present invention will be described with examples of aspects of digesting with a methylation-sensitive restriction enzyme in the digestion step.

In step (2) in the methylation analysis method of the present invention, i.e., in the coupling step, a mixture of DNA constructs can be obtained by concatenating the DNA fragment mixture obtained in said step (1), i.e., the digestion step, with ligase treatment.

As for each ligated site, the recognition site in each resulting DNA construct is regenerated; however, there is little possibility to meet the original base sequence located upstream of the recognition site and that located downstream of the recognition site, and then regenerated the original sequence (i.e., the pre-digestion recognition site and its surrounding base sequence).

Therefore, a new sequence that differs from original sequence is formed at the unmethylated recognition site which is cleaved with a methylation-sensitive restriction enzyme. On the other hand, at the recognition site that was the methylated recognition site, the same sequence as in the original sequence is maintained in each DNA construct since said recognition site and its surrounding sequence are maintained as intact in the digestion process.

In addition, in the ligation step of the present invention, a mixture of DNA fragments obtained in the digestion step may be ligated with ligase in the presence of double-stranded DNA adapters that can be ligated to both the ends thereof. When the ligase treatment is carried out in the presence of an excess amount of adapter phosphorylated at both the 5′ ends, the recognition site which was a non-methylated recognition site cleaved with methylation-sensitive restriction enzyme is been formed as a new sequence with one or more adapters sequences sandwiched between the fragments, which differs from original sequence. The adapter sequence can be uses in the analytical steps described below as a marker to assist in the identification of the cleaved site.

The number of the adapters sandwiched in the reproduced restriction enzyme recognition sequence is not particularly limited, but only one adapter sequence can be inserted by using an adapter in which the 5′ end of the oligonucleotide is not phosphorylated.

In addition, in the ligation step of the present invention, a group of desired DNA fragments may be fractionated from a mixture of DNA fragments obtained in step (1) prior to the ligase treatment. The fractionation methods may be included, for example, by gel filtration, ion-exchange resin or ion-exchange membrane, ultrafiltration, electrophoretic fractionation, alcohol precipitation, silicon-based filter, glass-based filter, and other DNA-binding resins or membranes (e.g., nitrocellulose-based, nylon-based, cationic, anti-DNA antibody, DNA-binding protein, methylated cytosine-binding protein, DNA-binding compound, intercalator), and DNA-binding molecules immobilized on resins or membranes.

In gel filtrations or ultrafiltration membrane fractionations, a group of DNA fragments with a particular molecular weight may be enriched and then ligated to polymerize those; an enrichment here includes the fractionations, e.g., 1) low molecular weight groups, 2) high molecular weight groups, 3) resin or membrane-bound fraction, 4) resin or membrane-bound fraction, etc., and then high molecular weight DNA may be obtained by ligating the DNA groups from one or more of the fractions.

The purpose of the fractionation process of the DNA fragment group of the present invention is to enrich and efficiently analyze the desired fraction in the DNA fragment group obtained by digestion with a restriction enzyme. Therefore, the purpose of the present process can be achieved even if it was carried out at any moment between the restriction enzyme digestion step and the ligation step of the DNA fragments. So that the similar effect can be expected even when the fractionation process is performed immediately after the process of restriction enzyme treatment process or just before the ligation.

The manipulation of fractionating a desired group of DNA fragments from a DNA fragment mixture can be carried out not only in the methylation analysis method of the present invention, but also in the restriction enzyme digestion step or the nuclease digestion step in the method for obtaining a group of DNA fragments of the present invention, which is described later. If the method for obtaining a group of DNA fragments of the present invention comprises one or more digestion steps, the fractionation procedures may be performed after one digestion step, fractionation procedures may be performed after two or more digestion steps, or fractionation procedures may not be performed.

Also, in the ligation step of the present invention, DNA amplification can be performed after said ligase treatment. Although the DNA amplification method is not particularly limited in this invention, the DNA amplification using strand substituted DNA polymerase (e.g., phi29 DNA polymerase) can be cited.

When a double-stranded DNA adapter whose 5′ ends are phosphorylated is used, DNA amplification can be performed in the conventional manner because the DNA fragment and the adapter are covalently linked by ligase treatment.

On the other hand, when double-stranded DNA adapters whose 5′ ends are not phosphorylated are used, the nick repaired DNA fragments which is obtained by linking the 5′ end of the double-stranded DNA adapter to the DNA fragments by ligase treatment, then phosphorylated by nucleotide kinase treatment, and then followed by ligase treatment, can be used as a template for strand displacement DNA polymerase. To fill the nick, PreCR Repair Mix (NEB) can be used as an example.

In the step (3) in the methylation analytical method of the present invention, i.e., in a sequencing step, the base sequence of each DNA construct included in the DNA constructs mixture obtained in said step (2), i.e., the ligation step is determined. Although the sequence can be determined by known means, such as sequencers, it is preferable to use next generation sequencers because the information that covers entire genome can be obtained.

In the step (4), i.e., analysis step, each recognition site of digestive restriction enzyme and its surrounding base sequence obtained in said step (3), i.e., sequencing step, it is determined whether said recognition sites are recognition sites that have not been cleaved by said restriction enzyme, or said recognition sites that has been regenerated by said ligation with ligase after cleavage with said restriction enzyme, by comparing to the known genomic sequence; thus each methylation status based on the information are to be determined.

In the analysis process of the present invention, methods of comparing each base sequence information obtained in the sequencing step to the known genome sequence is not particularly limited; however, as an example, it can be performed by mapping the base sequence between adjacent recognition sites to a known genome sequence, and then comparing the flanking sequence of at least one of the recognition sites of said adjacent recognition sites (i.e., for the sequence upstream of the recognition site, it comes under the upstream sequence of said recognition site; for the sequence downstream of the recognition site, it comes under the downstream sequence of said recognition site) to the mapped reference sequence.

More specifically, for example, it may be carried out with the methods including:
(a) a step of optionally selecting the first recognition site of digestive restriction enzyme(s) in each base sequence information obtained;
(b) a step of selecting the adjacent restriction enzyme recognition site downstream thereof as the second recognition site;
(c) a step of mapping the sequence between the first recognition site and the second recognition site to a known genomic sequence;
(d) a step of determining whether the second recognition site is a recognition site which has not been cleaved with the restriction enzyme or that is a recognition site which has been cleaved with the restriction enzyme and then regenerated by said ligation using ligase, by comparing the sequence downstream from the second recognition site to the mapped reference sequence;
(e) a step in which the adjacent restriction enzyme recognition site downstream of the second recognition site to be selected as the third recognition site, and then said step (c) and (d) are repeated (however, the first recognition site is to be read as the second recognition site, and the second recognition site is to be read as the third recognition site, hereinafter the same).

And, for example, it may be carried out with the methods including:
(a) a step of optionally selecting the first recognition site of a digestive restriction enzyme(s) for each base sequence information obtained;
(b) the step of selecting the adjacent restriction enzyme recognition site upstream thereof as the second recognition site;
(c) a step of mapping the sequence between the first recognition site and the second recognition site to a known genomic sequence;
(d) a step of determining whether the second recognition site is a recognition site which has not been cleaved with the restriction enzyme or that is a recognition site which has been cleaved with the restriction enzyme and then regenerated by said ligation using ligase, by comparing the sequence upstream from the second recognition site with the mapped reference sequence;
(e) a step in which the adjacent restriction enzyme recognition site upstream of the second recognition site is selected as the third recognition site, and then said step (c) and (d) are repeated (however, the first recognition site is to be read as the second recognition site, and the second recognition site is to be read as the third recognition site, hereinafter the same).

In step (d) of these methods described above, the sequence downstream (or upstream) of the second recognition site is to be compared with the corresponding mapped reference sequence; if it matches, it can be determined as a recognition site that was not cleaved by a digestive restriction enzyme(s) (e.g., methylation-sensitive restriction enzyme); accordingly, the recognition site in the DNA to be analyzed prior to the digestion can be determined as a recognition site (i.e., methylation recognition site) at which the particular cytosine involved in methylation sensitivity in the recognition site was methylated.

On the other hand, if it was not matched, it can be determined as a recognition site which is regenerated by said ligation with the ligase after cleaving with a digestive restriction enzyme (e.g., methylation-sensitive restriction enzyme); accordingly, both of said recognition sites (two sites) in the DNA to be analyzed prior to digestion that was the source of the regenerated recognition site can be determined to be a recognition site (i.e., non-methylated recognition site) in which particular cytosine involved in methylation sensitivity in the recognition sites are not methylated.

The effectiveness of using adapters is that only those containing adapter sequences can be extracted first from a large amount of data, and subsequent mapping to the genome can be efficiently performed using a computer. In other words, since only the sequence information near the non-methylated recognition site can be extracted first, the computational efficiency is greatly improved and the load on the computer is significantly reduced.

Thus, according to the present invention, the presence or absence of methylation at a specific position can be determined from each sequence information, and also the methylation rate at a specific position can be calculated from multiple base sequence information. That is, the methylation rate can be calculated by counting the sequence-read at a particular point out of the average number of sequence-read in all regions.

As described above, the present invention is a method of evaluating the methylation state of the restriction enzyme recognition site by means of digesting DNA to be analyzed (e.g., genomic DNA) with one or more digestive restriction enzymes (e.g., methylation-sensitive restriction enzymes) in combination, ligating them, and finally analyzing the base sequence of the DNA fragments concatenated with random ligation of each DNA fragment. Here we describe examples of the principle based on more specific embodiments with methylation-sensitive restriction enzymes as an example.

First of all, the DNA to be analyzed (e.g., human genomic DNA) is digested with one or more methylation-sensitive restriction enzymes.

Since the genomic DNA sample is a long-stranded, it may take time to digest it due to factors such as steric hindrance. Therefore, it is preferable to use restriction enzymes with long active half-lives and enzyme with high reaction temperatures. In the present invention, for example, the following restriction enzymes can be used; however, the present invention has the capability of using any restriction enzyme that meets the conditions described herein, and it is not limited to the restriction enzymes illustrated below.

| | |
|---|---|
| HinpII | (Optimal temperature: 37° C., G: CGC) |
| HpaII | (Optimal temperature: 37° C., C: CGG) |
| HpyCH4IV | (Optimal temperature: 37° C., A: CGT) |
| BstUI | (Optimal temperature: 60° C., CG: CG) |
| HhaI | (Optimal temperature: 37° C., GCG: C) |
| BstBI | (Optimal temperature: 65° C., TT: CGAA) |
| BssKI | (Optimal temperature: 60° C.,: CCNGG) |

For example, a restriction enzyme sensitive to methylation of cytosine in the recognition sequences, HinpII, HpaII or HpyCH4IV, may be used. These restriction enzymes have a long duration of activity at optimal temperatures and are stable in digestion reactions for several hours.

The higher the number of analyte sites in the analyte DNA, the better the resolution for the analysis of cytosine methylation in the -CpG- sequence in genomic DNA. To accomplish higher resolutions, a combination of multiple restriction enzymes which recognize different recognition sequences can be used.

For example, when the genomic DNA is to be digesting with two or three kinds in HinPII, HpaII and HpyCH4 IV with a combination, the 5′ protruding ends of the resulting fragments are all to be -CpG- (5′-CG-XXXX-3′) (X is any base); so that the sticky ends of the DNA fragments that have been cleaved with the different restriction enzymes can be even ligated together by ligation treatment without selecting their ligating partners. The concatenated DNA obtained by ligating the protruding ends with different base sequence, which are produced with different restriction enzymes, cannot no longer be re-cleaved with the restriction enzymes originally be used; however, the sequence analysis will not be affected.

Methylation-sensitive restriction enzymes that produce blunt ends can also be used in this method. For example, even when a restriction enzyme that produces fragments with blunt ends is used in combination with a restriction enzyme that generates sticky ends, ligation treatment can be performed without any particular pretreatment since the DNA fragments produced by each restriction enzyme have their own ligating partner in the ligation.

The restriction enzymes used in the digestion process of the present invention do not require a common nucleic acid sequence at the protruding ends, and as shown in the examples below, it is not essential to use a set of restriction enzymes that produce common protruding ends; so that methylation-sensitive restriction enzymes can freely be used in combination since the sticky ends produced by a particular restriction enzyme can be ligated themselves. Thus, this method has a significant advantage in that enzyme can freely combine to use when compare to the conventional methods. In the conventional methylation analysis using restriction enzyme required a combination of restriction enzymes with the same recognition sequence; such as HpaII, a methylation-sensitive restriction enzyme; MspI, a methylation-insensitive restriction enzyme. However, such a combination of restriction enzymes is very rare, and the target region to analyze the methylation is restricted by their recognition sequence of the restriction enzymes, so that the gene regions or the plant genomes without this recognition sequence could not even be analyzed. The method of the present invention solves this issue and not only enables the application of conventional restriction enzyme-based methylation analysis methods to plant genomes, but also dramatically improves the resolution of the analysis; furthermore, it allows us to use a number of restriction enzymes in combination depending on the base sequence of the region to be analyzed.

In addition, it is possible to use a combination of methylation-insensitive restriction enzymes in the processing or the pre-treatment of the genome with above-mentioned restriction enzymes. Even in such a case, methylation-insensitive restriction enzymes with recognition sequences which is different from that of methylation-sensitive restriction enzymes can be appropriately used simultaneously since it does not influence the methylation analysis of the site subject to methylation analysis. For example, for the purpose of promoting a complete digestion of genomic DNA, pre-digestion can be performed with methylation-insensitive restriction enzyme with high optimal temperature at which the conformation of the genomic DNA can be destabilized, and then can be digested with methylation-sensitive restriction enzymes.

Next, we illustrate the case of using HinPII and HpaII as methylation-sensitive restriction enzymes as an example. Digestion of DNA with HinPII and HpaII produces the following DNA fragments.
1) DNA fragments with HinPII sites at both the ends;
2) DNA fragments with HpaII sites at both the ends;
3) a DNA fragment with one HpaII site and the other HinPII

When the ligation is performed for the DNA fragment mixture containing those above, the concatenated long-chain DNA illustrated below is produced.

5′---HinPII-------HinPII-p-HinPII-------HpaII-p-HinPII-------HpaII-p-HpaII----3′

The sticky ends produced by each restriction enzyme are denoted by its enzyme name. Also, “-p-” denotes the ligation-bound site.

In addition, the sequences produced with each combination of said fragments 1) to 3) (a combination of 3′ ends and 5′ ends) are shown in Table 2.

TABLE 2

| 3′ end & 5′ end | Sequence to be produced | | | |
|---|---|---|---|---|
| Combination | G C G C | | | |
| of 1) & 1) | | | | |
| Combination | G C G G | | | |
| of 1) & 2) | | | | |
| Combination | G C G G (*1) | G C G C (*2) | | |
| of 1) & 3) | | | | |
| Combination | C C G C | | | |
| of 2) & 1) | | | | |
| Combination | C C G G | | | |
| of 2) & 2) | | | | |
| Combination | C C G G (*1) | C C G C (*2) | | |
| of 2) & 3) | | | | |
| Combination | C C G C (*1) | G C G C (*2) | | |
| of 3) & 1) | | | | |
| Combination | C C G G (*1) | G C G G (*2) | | |
| of 3) & 2) | | | | |
| Combination | C C G G (*3) | C C G C (*4) | G C G G (*5) | G C G C (*6) |
| of 3) & 3) | | | | |

(*1): in case of binding at HpaII side of (3);
(*2): in case of binding at HinPII side of (3);
(*3): in case that the (3) at 3′-end is HpaII site, and (3) at 5′-end is HpaII site;
(*4): in case that the (3) at 3′-end is HpaII site, and (3) at 5′-end is HinpII site;
(*5): in case that the (3) at 3′-end is HinpII site, and (3) at 5′-end is HpaII site;
(*6): in case that the (3) at 3′-end is HinpII site, and (3) at 5′-end is HinpII site;

Thus, the DNA fragments produced from each restriction enzyme can generate the recognition sequences which differ from that produced with the same species at both the ends; however, the protruding bases produced with HinPII and HpaII both have -CpG-, so that the DNA fragments produced with the different restriction enzymes can also be ligated together by ligation. Thus, the DNA fragments are to be polymerized by ligating the partners which can be ligated each other.

The resulting concatenated long-chain DNA can be handle as a DNA sample that can be analyzed with conventional sequencers. In general, the analyte DNA is to be fragmentated with ultrasonication or enzyme digestion (cleave at the desired site), and ligate the adapters provided from the manufacturer of each sequencing device, and then analyze the base sequence with the sequencer.

The resulting sequence information of individual DNA is to be mapped to the genomic DNA sequence information to be analyzed, thereby the cleaving sites with used restriction enzymes are to be clarified.

On the other hand, when the restriction enzyme recognition sites of used restriction enzyme remain intact in the base sequence data of the sequence analysis, the cytosine in the -CpG- sequence of the site is to be evaluate as a methylated and therefore resistant against the restriction enzyme digestion performed.

Thus, whichever the DNA fragment which is cut or uncut with particular restriction enzyme, the methylation of cytosine in the recognition sequence of used restriction enzyme can be analyzed by mapping the DNA fragments to the reference genome sequence information.

### <Method for Obtaining a DNA Fragment Group>

The present invention includes methods for obtaining a group of DNA fragments comprising only specific DNA.

A feature of the method for obtaining a DNA fragment group in the present invention as described in detail below is to use a combination of methylation-sensitive restriction enzyme and methylated-insensitive restriction enzyme, wherein the recognition sequences of both are identical and produces protruding ends; and to use an adapter(s) to be ligated to the protruding ends in the first ligation step carried out after the first digestion step performed with a methylation-sensitive restriction enzyme, wherein the adapter has sequence that does not regenerate the original recognition sequence.

According to the method for obtaining DNA fragments of the present invention, as described below:

A group of DNA fragments comprising of only DNA fragments (hereinafter referred to as double-ended methylated cytosine DNA fragments) in which methylated cytosine is present at both the protruding ends, or a group of DNA fragments comprising only DNA fragments (hereinafter referred to as double-ended cytosine DNA fragments) in which cytosine (i.e., unmethylated cytosine) is present at both the protruding ends, can be obtained. The methylation status of these DNA fragments can be determined with focus on the specific DNA fragments (i.e., double-ended methylated cytosine DNA fragments or double-ended cytosine DNA fragments) by determining the base sequence of a long concatenated DNA produced with ligase treatment. Accordingly, since the DNA fragments to be sequenced is concentrated, significant increasement of sequencing efficiency, great shortening of analysis time and cost savings can be expected.

### [Method for Obtaining DNA Fragments Consisting of Only Double-Ended Methylated Cytosine DNA Fragments]

The first method for obtaining a DNA fragment group consisting of only double-ended methylated cytosine DNA fragment of the present invention (hereinafter referred to as the double-ended methylated cytosine DNA obtaining method) includes:
(1) digesting a DNA to be analyzed with a methylation-sensitive restriction enzyme whose recognition site includes methylated cytosine or cytosine to be methylated and which generates a cohesive end (the first digestion process);
(2) ligating labeled adapters which do not generate a recognition site of the methylation-sensitive restriction enzyme, to both ends of the DNA fragments obtained in step (1) (ligation process);
(3) digesting the labeled DNA constructs obtained in step (2) with a methylation-insensitive restriction enzyme which recognizes the same recognition site as the methylation-sensitive restriction enzyme and which generates a cohesive end (the second digestion process); and
(4) removing only the labeled DNA fragments from the mixture of DNA fragments obtained in step (3) using a binding partner specific to the label, to obtain a DNA fragment group consisting of DNA fragments in which methylated cytosines are included in both cohesive ends at both ends (removing process).

The second method for obtaining double-ended methylated cytosine DNA includes:
(1) digesting a DNA to be analyzed with a methylation-sensitive restriction enzyme whose recognition site includes methylated cytosine or cytosine to be methylated and which generates a cohesive end (the first digestion process);
(2) converting both ends of the DNA fragments obtained in step (1) into blunt ends in the presence of labeled deoxynucleoside triphosphate (fill-in process);
(3) digesting the labeled DNA fragments obtained in step (2) with a methylation-insensitive restriction enzyme which recognizes the same recognition site as the methylation-sensitive restriction enzyme and which generates a cohesive end (the second digestion process); and
(4) removing only the labeled DNA fragments from the mixture of DNA fragments obtained in step (3) using a binding partner specific to the label, to obtain a DNA fragment group consisting of DNA fragments in which methylated cytosines are included in both cohesive ends at both ends (removing process).

The third method for obtaining double-ended methylated cytosine DNA includes:
(1) digesting a DNA to be analyzed with a methylation-sensitive restriction enzyme whose recognition site includes methylated cytosine or cytosine to be methylated and which generates a cohesive end (the first digestion process);
(2) ligating stem-loop adapters which do not generate a recognition site of the methylation-sensitive restriction enzyme, to both ends of the DNA fragments obtained in step (1) (the first ligation process);
(3) digesting the DNA constructs obtained in step (2) with a methylation-insensitive restriction enzyme which recognizes the same recognition site as the methylation-sensitive restriction enzyme and which generates a cohesive end (the second digestion process);
(4) ligating nuclease-resistant labeled adapters whose 5′-end shows nuclease resistance and which generate the recognition site of the methylation-insensitive restriction enzyme, to each cohesive end of the DNA fragments obtained in step (3) (the second ligation process); and
(5) treating the DNA constructs obtained in step (4) with a single-strand-specific endonuclease followed by a combination of double-strand-specific and single-strand-specific endonucleases, to completely digest only DNA fragments to which the stem-loop adapters are ligated, and to obtain a DNA fragment group consisting of DNA fragments to which the nuclease-resistant labeled adapters are ligated at both ends thereof (the third digestion process).

In the method for obtaining double-ended methylated cytosine DNA of the present invention, two restriction enzymes with identical recognition sequences are used.

In the first digestion process, methylation-sensitive restriction enzyme(s) [hereinafter referred to as methylation-sensitive restriction enzyme (MS-restriction enzyme)] whose recognition sequence includes methylated cytosine or potentially methylated cytosine, and produces a protruding end, is(are) used; and
in the second digestion process, methylation-insensitive restriction enzyme(s) [hereafter referred to as methylation-insensitive restriction enzymes (MI restriction enzymes)] which recognizes the same recognition sequence as said methylation-sensitive restriction enzyme, and generates a protruding end, is(are) used.

MS restriction enzymes and MI restriction enzymes that can be used in the method of the present invention includes, for example, a combination of HpaII (MS restriction enzyme) and MspI (MI restriction enzyme).

In the first method to obtain DNAs with methylated cytosine at both ends of the present invention, the analyte DNA is digested with a methylation-sensitive restriction enzyme. All of the DNA fragments obtained here are to be the DNA fragments in which cytosine is present at the both protruding ends. After ligating the labeled adapters (e.g., biotinylated adapters, digoxigenin-labeled adapters), which do not regenerate the recognition sequence of methylation-sensitive restriction enzyme(s), to the both ends of the resulting DNA, cleave the recognition sequence containing methylated cytosine by digesting the resulting labeled DNA constructs, which have the labeled adapters at the both ends, with methylation-insensitive restriction enzyme(s); thus the DNA further fragmented.

Since the methylation-insensitive restriction enzyme cleaves the methylated recognition sequence within the labeled DNA construct, the resulting DNA fragment is to be a mixture of:
(1) uncleaved labeled DNA constructs with labeled adapters at the both ends;
(2) DNA fragments with labeled adapter at one end, and with the protruding end containing methylated cytosine at another end; and
(3) DNA fragments with protruding ends containing methylated cytosines at the both ends.

Thus, a group of DNA fragments (methylated cytosine DNA fragment at the both ends) consisting only of the DNA fragments (3) in which methylated cytosine is present at the both ends can be obtained by contacting the said DNA fragments with a specific binding partner (e.g., avidin beads, avidin columns, anti-digoxigenin antibody beads, or anti-digoxigenin antibody columns) of said label (e.g., avidin, anti-digoxigenin antibody) to remove the DNA fragments (1) and (2) to which the labeled adapter is linked.

In the second method to obtain methylated cytosine DNA at both ends of the present invention, the analyte DNA is digested with a methylation-sensitive restriction enzyme. All of the DNA fragments obtained herein are to be the DNA fragments in which cytosines are present at the both protruding ends. After filling-in the protruding ends of the resulting DNA fragments in the presence of labeled deoxynucleoside triphosphates (e.g., biotinylated deoxynucleoside triphosphates, digoxigenin-labeled deoxynucleoside triphosphates), cleave the recognition sequence containing methylated cytosine by digesting the resulting DNA fragments, which are blunted and labeled at the both ends, with methylation-insensitive restriction enzyme(s); and further fragment the DNAs.

Since the methylation-insensitive restriction enzyme cleaves the methylated recognition sequence within the labeled DNA construct, the resulting DNA fragments are to be a mixture of:
(1) uncleaved DNAs with blunted and labeled adapters at the both ends;
(2) DNA fragments with blunted and a labeled adapter at one end, and protruding end containing methylated cytosines at the another end; and
(3) DNA fragments with protruding ends containing methylated cytosines at the both ends.

Thus, a group of DNA fragments (methylated cytosine DNA fragment at the both ends) consisting only of the DNA fragments (3) in which methylated cytosine is present at the both ends can be obtained by contacting the said DNA fragments with a specific binding partner (e.g., avidin beads, avidin columns, anti-digoxigenin antibody beads, or anti-digoxigenin antibody columns) of said label (e.g., avidin, anti-digoxigenin antibody) to remove the DNA fragments (1) and (2) to which the labeled adapter is linked.

In the first or the second method in the present invention to obtain DNA fragments with methylated cytosine at the both ends or in the method in the present invention to obtain a group of DNA fragment consisting solely of DNA fragments with cytosine at the both ends described below, the known combination using affinity such as biotin/avidin, digoxigenin/anti-digoxigenin antibodies as their specific partners of labeling adapters or labeled deoxynucleoside triphosphates can be used.

In the third method in the present invention to obtain methylated cytosine DNA at the both ends, the DNA is fragmented by digesting the analyte DNA with methylation-sensitive restriction enzyme(s). All of these DNA fragments are DNA fragments in which cytosine is present at the both protruding ends. After ligating stem-loop adapter (labeling is not specifically required) which do not regenerate the recognition sequence of the methylation-sensitive restriction enzyme(s) to the both ends of the resulting DNA, cleave the recognition sequence containing methylated cytosine by digesting the resulting labeled DNA constructs which have the labeled adapters at the both ends with methylation-insensitive restriction enzyme(s); consequently, the DNA further fragmented. Since the methylation-insensitive restriction enzyme cleaves the methylated recognition sequence within the stem-loop adapter linked DNA construct, the resulting DNA fragment is to be a mixture of:
(1) uncleaved DNA constructs with stem-loop adapters at the both ends;
(2) DNA fragments with stem-loop adapter at one end, and with protruding end containing methylated cytosine at another end; and
(3) DNA fragments with protruding ends containing methylated cytosines at the both ends.

Subsequently, ligate the nuclease-resistant labeled adapters, wherein the 5′ end exhibits nuclease resistance and regenerates the recognition sequence of said methylation-insensitive restriction enzyme, to each protruding end of said DNA fragment mixture.

Since both the ends of said DNA construct (1) or a one end of said DNA fragment (2) is/are linked to the stem-loop adapter(s), said nuclease-resistant labeled adapter does not further linked to, and consequently, a mixture of DNA construct consisting:
(1) uncleaved DNA construct which has stem-loop adapters at the both ends;
(2′) DNA construct with stem-loop adapter at one end, and a nuclease-resistant labeled adapter is linked to the protruding end containing methylated cytosine at another end; and
(3′) a mixture of DNA construct wherein the nuclease-resistant labeled adapter linked to the protruding ends containing methylated cytosines at the both ends;
can be obtained.

By treating the resulting DNA construct mixture with a single-strand specific endonuclease (e.g., Mung bean nuclease or S1 nuclease; reactive in acidic) which has endonuclease specificity to ssDNA, digest the stem-loop structure region comprising the ssDNA region and the dsDNA region which DNA constructs (1) and (2′) possess. Subsequently, said DNA construct (1) and (2′) can be completely digested by treating with a combination of double-strand specific exonuclease (e.g., λ-exonuclease with 5′→3′ exonuclease activity) and single-strand specific exonuclease (e.g., exonuclease I) (reactive in alkaline); on the other side, a group of DNA fragment consisting only of the DNA construct (3′) can be obtained since the DNA construct (3′), which has nuclease-resistant labeled DNA at the both ends, remains as an undigested.

Moreover, since both À exonuclease and exonuclease I prefer alkaline solutions as their optimum pH circumstances, it can be used simultaneously (i.e., simultaneous digestion) in the same buffer [i.e., in NEBuffer 4 or CutSmart buffer (both from NEB: New England Biolabs)].

In addition, since the property of reaction solution for single strand specific endonuclease and that for a combination of double strand specific exonuclease and single strand specific exonuclease (e.g., exonuclease I) is different, the DNA after treating with single strand specific endonuclease can be purified by using conventional methods [e.g., QIAmp™ DNA mini kit (QIQGEN)]. Furthermore, the remaining undigested DNA construct (3′) can also be purified by conventional methods (e.g., QIAamp™ DNA Mini kit (QIAGEN) considering subsequent enzymatic treatment.

Subsequently, when the DNA fragments are digested with said methylation-insensitive restriction enzyme, the labeled adapter can be cleaved out from each DNA construct because the recognition sequence of the methylation-insensitive restriction enzyme has been regenerated by the prior linkage of the methylated cytosine-containing protruding end with the nuclease resistance labeled adapter. The resulting digest may be contacted with a specific binding partner of said label (e.g., contacted with avidin beads or avidin columns) to remove the nuclease-resistant labeled adapters, and then a group of DNA fragments (DNA fragments with methylated cytosine at the both ends) consisting only of DNA fragments containing methylated cytosine at the both ends can be obtained.

As for the DNA fragments with methylated cytosine at the both ends obtained by the method for obtaining DNA with methylated cytosine at the both ends described in the first to third methods in the present invention, a state of methylation of the DNA fragments with methylated cytosine at the both ends can be determined by sequencing of the DNA after treating with ligase to yield long-strand concatenated DNA.

A method for obtaining DNA fragments consisting solely of bi-terminal cytosine DNAs]

A method for obtaining DNA fragments consisting solely of bi-terminal cytosine DNAs (hereinafter referred to as the “method for obtaining bi-terminal cytosine DNA”) in

the present invention comprising:
(1) a step for digesting DNA to be analyzed with a methylation-sensitive restriction enzyme whose recognition site includes methylated cytosine or cytosine to be methylated and which generates a cohesive end (the first digestion process);
(2) a step for ligating nuclease-resistant labeled adapters whose 5′-end shows nuclease resistance and which have a restriction enzyme recognition site consisting of 8 nucleotides or more and do not generate a recognition site of the methylation-sensitive restriction enzyme, to both ends of the DNA fragments obtained in step (1) (the first ligation process);
(3) a step for digesting the DNA constructs obtained in step (2) with a methylation-insensitive restriction enzyme which recognizes the same recognition site as the methylation-sensitive restriction enzyme and which generates a cohesive end (the second digestion process);
(4) a step for ligating stem-loop adapters to both ends of the DNA fragments obtained in step (3) (the second ligation process);
(5) a step for treating the DNA constructs obtained in step (4) with a single-strand-specific endonuclease followed by a combination of double-strand-specific and single-strand-specific endonucleases, to completely digest only DNA fragments to which the stem-loop adapters are ligated, and to obtain a DNA fragment group consisting of DNA fragments to which the nuclease-resistant labeled adapters are ligated at the both ends thereof (the third digestion process).

In the method for obtaining bi-terminal cytosine DNA in the present invention, the DNA is fragmented by digesting the analyte DNA with methylation-sensitive restriction enzyme. All the DNA fragments in this fraction are composed of the DNA fragments in which unmethylated cytosine is present in the both CG sequences at the protruding ends. A nuclease-resistant labeled adapter (e.g., nuclease-resistant biotinylated adapter, nuclease-resistant digoxigenin-modified adapter), which includes a restriction enzyme recognition sequence consisting of 8 nucleotides or more in length and not regenerating the recognition sequence of said methylation-sensitive restriction enzyme, is ligated to the protruding ends of the resulting DNA fragment. The DNA is further fragmented by digesting the resulting DNA construct, which has a nuclease-resistant labeled adapter linked to both the ends, with methylation-insensitive restriction enzyme(s).

Since the methylation-insensitive restriction enzyme cleaves the methylated recognition sequence within the nuclease-resistant labeled DNA construct, the resulting DNA fragments are to be a mixture of:
(1) DNA constructs that have not been cleaved but have the nuclease-resistant labeled adapters at both ends;
(2) DNA fragments with protruding ends that has nuclease-resistant labeled adapter at one end, and has the restriction enzyme recognition sequence containing unmethylated cytosine at another end; and
(3) DNA fragments with the restriction enzyme recognition sequence at both the protruding ends in which unmethylated cytosines are present.

Each protruding end of the resulting DNA fragments are then ligated with a stem-loop adapter (the labeling is not required. And also, whether or not it can regenerate the recognition sequence is not especially required.)

Since the both ends of the DNA construct (1) or one end of the DNA fragment (2) is/are ligated to a nuclease-resistant labeled adapter(s), the stem-loop adapter is not further conjugated; consequently, a mixture of DNA construct comprising:
(1) DNA construct in which nuclease-resistant labeled adapters are ligated to the both ends;
(2′) DNA construct in which a nuclease-resistant labeled adapter is ligated to one end, and a stem-loop adapter is ligated to the protruding end containing unmethylated cytosine at another end; and
(3) a mixture of DNA construct in which stem-loop adapters are ligated to the protruding ends containing unmethylated cytosines at both the ends, can be obtained.

By treating the resulting DNA construct mixture with a single-strand specific endonuclease which has endonuclease specificity to ssDNA (e.g., Mung bean nuclease or S1 nuclease; reactive solution is acidic), decompose the stem-loop structural region consisting of the ssDNA region and the dsDNA region which said DNA construct (2′) and (3′) possess.

Subsequently, said DNA constructs (2′) and (3′) can be completely degraded by treating with a combination of a double strand-specific exonuclease (e.g., λ exonuclease with 5′→3′ exonuclease activity) and a single strand-specific exonuclease (e.g., exonuclease I) (reactive solution is alkaline).

On the other hand, since only the DNA constructs (1) (both ends are derived from protruding ends containing cytosines) with nuclease-resistant labeled adapters at the both ends remain undigested, the DNA fragment group consisting solely of the DNA construct (1) can be obtained.

In addition, since both À exonuclease and exonuclease I prefer alkaline solution as their optimal pH, those can be use in the same reaction solution (e.g., NEBuffer 4 or CutSmart buffer; both available from NEB) simultaneously (i.e., double digestion). In addition, since a reaction solution for single-strand specific endonuclease and that for a mixture of double strand-specific exonuclease and single strand-specific exonuclease (e.g., exonuclease I) is different in their property, DNA after treating with single-strand specific endonuclease may be purified by using the conventional methods [e.g., QIAamp™ DNA Mini kit (QIAGEN)].

The remaining undigested DNA construct (1) may also be purified with conventional methods (e.g., QIAamp™ DNA Mini kit (QIAGEN) considering subsequent enzymatic treatment.

Subsequently, the DNA fragments can then be digested with restriction enzymes that recognize the restriction enzyme recognition sequences consisting of 8 nucleotides or more in length within the nuclease-resistant labeling adapter to cleave off the labeling adapter from each DNA construct. The obtained digested product may be brought into contact with a specific binding partner (e.g., avidin, anti-digoxigenin antibodies beads or anti-digoxigenin antibodies column) of said label (e.g., contact with avidin beads, avidin columns, anti-digoxigenin antibody beads or anti-digoxigenin antibody columns), and thus a DNA fragment group (unmethylated cytosine DNA fragments at the both ends) comprising only DNA fragments having unmethylated cytosine (within a CpG sequence) near both the protruding ends can be obtained.

The bi-terminal unmethylated cytosine DNA obtained by the method for obtaining bi-terminal unmethylated cytosine DNA in the present invention may be treated with ligase to make a long concatenated DNA, and then the methylation status thereof can be determine by analyzing its base sequence.

### <<DNA Amplification Methods Available in the Present Invention>>

The structure flexibility of double-stranded DNA changes with the presence of divalent cations such as magnesium.

For example, double-stranded DNA tends to form a linear structure in the presence of magnesium ion while the DNA strand is structurally flexible at lower concentration thereof. So that, either a long-chain or a self-closing circular structure can be formed depending on the conditions when the DNA fragment is ligated.

Therefore, when DNA is amplified using a long double-stranded DNA as a template, a strand-replacement DNA polymerase (e.g., phi 29 DNA polymerase) provided in, for example, the Illustra™ GenomiPhi™ DNA Amplification Kit (GE Healthcare) can be used; and when DNA amplification is desired using a circular structured DNA as a template, DNA amplification using a strand-replacement DNA polymerase (e.g., phi 29 DNA polymerase) can be carried out using such as the Illustra™ TemplatePhi™ DNA Amplification Kit (GE Healthcare), etc., which is widely used for amplification of plasmid DNA based on the Rolling Circle Amplification (RCA) method. The DNA amplification method described in the present invention, together with the DNA fragment fractionation method described in the present invention, can be selected as desired, and any combination of methods can achieve the object of the present invention.

### Example 1: Method for Determination of Methylation Rate

<A Preparation of Concatenated Long-Chain DNA (High Molecular Weight DNA with Random Conjugation) for Next-Generation Sequencer Analysis (in Case without DNA Amplification Step)>

Genomic DNA was purified from human fibroblast WI-38 (10×10E6) using a genomic DNA purification kit QIAamp DNA Mini Kit (QIAGEN); the treatment time with Proteinase K in this purification step was carried out at 56° C. for 4 hours.

Before eluting DNA from the purification column of the kit, the purification column was dried under reduced pressure for 5 minutes to remove residual alcohol, and then DNA was eluted with 40 μL of 1× CutSmart Buffer (New England Biolabs).

Take an aliquot corresponding to 100 ng of the purified DNA; adjust the total volume to 50 μL with 1× CutSmart Buffer; add 0.4 units each of HpaII (New England Biolabs) and HhaI (New England Biolabs), respectively; and then incubated at 37ºC for 4 for digestion. The recognition sequence of HpaII is C ↓ CGG, and produces a sticky end with an overhang at the 5′ end; the recognition sequence of HhaI is GCG ↓ C, and produces a sticky end with an overhang at the 3′ end.

The sticky ends generated by HpaII, or sticky ends generated by HhaI can be ligated with each other within the same population; however, the sticky ends generated by HpaII and those by HhaI are not to be ligated together.

The DNA containing solution was obtained from the resultant DNA-digestion solution by using a MinElute™ PCR Purification Kit (QIAGEN) by eluting the DNA from the purification column with 10 μL of DNA elution buffer according to the manufacturers' instructions. The collected DNA was then ligated using a Quick Ligation Kit (New England Biolabs), so that the randomly concatenated DNA fragments (long-strand concatenated DNA) was prepared.

For the solution containing the concatenated long-chain DNA prepared in said procedure, the concatenated long-chain DNA was purified using a QIAamp™ DNA Mini Kit (QIAGEN). As for the analysis using the next generation sequencer (Illumina), a sample for sequencing analysis was prepared from the concatenated long-chain DNA purified in said procedure according to the procedure recommended by the manufacturer, and then applied to the next generation sequencer for sequencing analysis.

<Preparation of Concatenated Long-Chain DNA (High Molecular Weight DNA with Random Ligation) for the Next Generation Sequencing Analysis (in Case DNA Amplification Step is Included)>

The genomic DNA was purified in the same manner as described above and then digested with restriction enzymes; however, when a DNA amplification was carried out, a Takara DNA Ligation Kit LONG (Takara Bio) was used for random ligation of the DNA fragments. And then the long-strand concatenated DNA was purified using the QIAamp™ DNA Mini Kit (QIAGEN) in the same manner as described above. In the case of amplifying said purified DNA, an aliquot of the purified DNA solution obtained in said procedure was taken and amplified the DNA according to the instruction manual provided in the illustra GenomiPhi™ V2 Kit (GE Healthcare Japan).

The amplified DNA was purified using a QIAamp™ DNA Mini Kit™, and then the samples for the analysis were prepared according to the same procedure recommended by manufacture of the next generation sequencer (Illumina), and carried out a sequencing analysis.

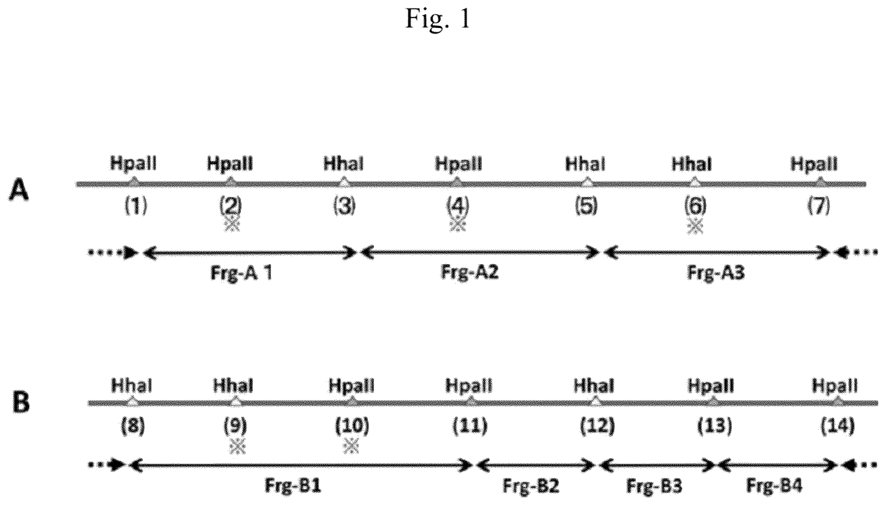

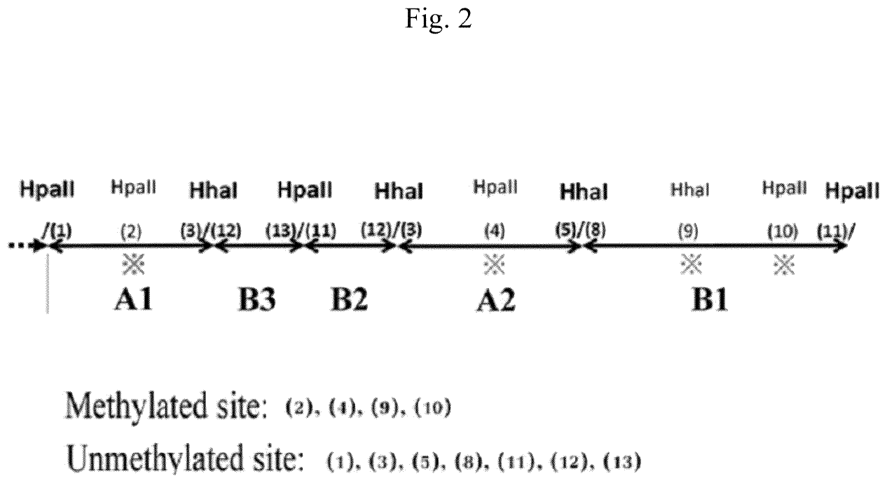

To support understanding of the structure of the obtained long-strand concatenated DNA, the structure of the genomic DNA before digestion with a methylation-sensitive restriction enzyme is illustrated in FIG. 1; the structure of the long-strand concatenated DNA obtained by ligation after digestion with said restriction enzyme is shown in FIG. 2.

FIG. 1 is an explanatory diagram schematically showing the structures of partial regions A and B of genomic DNA, showing the recognition sites [(1)-(14)] of methylation-sensitive restriction enzymes HpaII and HhaI, and a recognition site in which cytosine is methylated in a CpG sequence is indicated by the symbol “*”.

Digestion of genomic DNA with the methylation-sensitive restriction enzymes HpaII and HhaI results in cleavage by both restriction enzymes only at recognition sites where cytosine in the CpG sequence is not methylated (i.e., unmethylated sites), resulting in fragments A1, A2, and A3 from subregion A and fragments B1, B2, B3, and B4 from subregion B.

Since the ends of each DNA fragment are either sticky ends generated by HpaII or sticky ends generated by HhaI, the sticky ends generated by HpaII or those generated by HhaI are ligated in each of themselves to produce a concatenated long-chain DNA in which fragments A1, B3, B2, A2 and B1 are ligated in this order, for example, as shown in FIG. 2.

For example, the ligation of fragment A1 with fragment B3 is produced by ligating the sticky end from unmethylated HhaI site (3) with the sticky end from unmethylated HhaI site (12).

Focusing on the HpaII recognition site and HhaI recognition and their upstream and downstream sequences shown in FIG. 2, the methylated recognition sites, i.e., the upstream and downstream sequences of HpaII (2), HpaII (4), HhaI (9), and HpaII (10), respectively, retain their original base sequences.

On the other hand, the recognition sites [e.g., HhaI site (3)/(12) regenerated by ligation of fragment A1 and fragment B3] regenerated by ligating the different DNA fragments are all derived from unmethylated sites, and the upstream and downstream sequences of the regenerated recognition sites are derived from different DNA fragments (e.g., at the HhaI site (3)/(12), fragment A1 and fragment B3), respectively.

Accordingly, in the long-strand concatenated DNA obtained by ligation, by mapping each base sequence upstream and downstream of each recognition site of the restriction enzyme used for the digestion of the genome DNA to a human genome reference sequence, the methylation state of cytosine in each recognition sequence in the original genome sequence can be thus determined.

### <Identification of Methylated and Unmethylated Sites>

In this embodiment, the methylation state was determined based on DNA sequence information output from the next generation sequencer. The DNA sequence information obtained was mapped onto the human genome reference sequence using conventional software according to the conventional methods, and presence or absence of methylation in the restriction enzyme recognition sites targeted in the analysis was discriminated. In particular, since the cleaved restriction enzyme recognition site is randomly ligated to other DNA fragments not originally be adjacent, when mapping to a reference sequence is performed, either one of the strand of upstream or of downstream of the restriction enzyme recognition site can only be mapped to the reference sequence.

In case that only one of DNA fragment can be mapped across the restriction enzyme recognition sequence, the restriction enzyme recognition site was counted as an unmethylated site because the restriction enzyme recognition site was cleaved by a methylation-sensitive restriction enzyme.

When cytosine in the CpG sequence of a restriction enzyme recognition site is methylated, it is not to be cleaved by the methylation-sensitive restriction enzyme, so that the original base sequence is retained both in the upstream and in the downstream of the DNA fragments across the restriction enzyme recognition site; so that, all the base sequences can be mapped to the same region on the genome reference sequence. Such restriction enzyme recognition sites were counted as methylation sites.

This mapping process is performed on all the data output from the next generation sequencer and accumulate multiple methylation information about 10 times on average for a particular restriction enzyme recognition site.

In case that the mapping was performed for one restriction enzyme recognition sequence for 10 times in average, and when five of the lead sequence information in the analyte DNA fragment, i.e., upstream and downstream base sequences flanking the restriction enzyme recognition site, were retained intact as the original genome base sequence, we have determined a methylation rate of the restriction enzyme recognition site as 50% meaning five tenth.

And, in case that the mapping was performed for a particular restriction enzyme recognition site for 10 times in average, and when two of the lead sequence information included upstream and downstream base sequences flanking the restriction enzyme recognition site in the analyte DNA fragment were retained intact as the original genome base sequence, we have determined a methylation rate of the restriction enzyme recognition site as 20%, meaning two tenths.

### Example 2

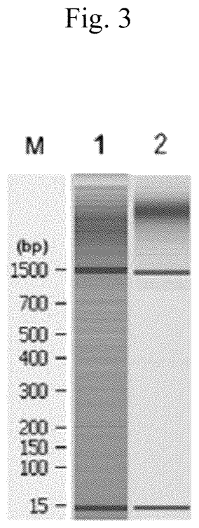

A DNA fragment mixture obtained by digesting genomic DNA with methylation-sensitive restriction enzymes HpaII and HhaI, and a polymerized long-strand concatenated DNA obtained by ligating the DNA fragment mixture were obtained by repeating the procedure of Example 1 in the present invention except that the human fibrosarcoma HT-1080 strain is used instead of the human fibroblast WI-38, and electrophoresis was performed. The results are shown in FIG. 3; lane 1 shows a mixture of DNA fragments obtained by digesting with HpaII and HhaI; lane 2 shows a polymerized long-strand concatenated DNA obtained by ligating the mixture of DNA fragments.

The present invention is applicable for the use of DNA methylation analysis.

1-9. (canceled)

10. A method for obtaining a DNA fragment group, comprising:
(1) digesting a DNA to be analyzed with a methylation-sensitive restriction enzyme whose recognition site includes methylated cytosine or cytosine to be methylated and which generates a cohesive end;
(2) ligating labeled adaptors which do not generate a recognition site of the methylation-sensitive restriction enzyme, to both ends of the DNA fragments obtained in step (1);
(3) digesting the labeled DNA constructs obtained in step (2) with a methylation-insensitive restriction enzyme which recognizes the same recognition site as the methylation-sensitive restriction enzyme and which generates a cohesive end; and
(4) removing only the labeled DNA fragments from the mixture of DNA fragments obtained in step (3) using a binding partner specific to the label, to obtain a DNA fragment group consisting of DNA fragments in which methylated cytosines are included in both cohesive ends at both ends.

11. A method for obtaining a DNA fragment group, comprising:
(1) digesting a DNA to be analyzed with a methylation-sensitive restriction enzyme whose recognition site includes methylated cytosine or cytosine to be methylated and which generates a cohesive end;
(2) converting both ends of the DNA fragments obtained in step (1) into blunt ends in the presence of labeled deoxynucleoside triphosphate;
(3) digesting the labeled DNA fragments obtained in step (2) with a methylation-insensitive restriction enzyme which recognizes the same recognition site as the methylation-sensitive restriction enzyme and which generates a cohesive end; and
(4) removing only the labeled DNA fragments from the mixture of DNA fragments obtained in step (3) using a binding partner specific to the label, to obtain a DNA fragment group consisting of DNA fragments in which methylated cytosines are included in both cohesive ends at both ends.

12. A concatenated long-chain DNA that holds methylation information, obtained by multiple-ligating the DNA fragment group consisting of DNA fragments in which methylated cytosines are included in both cohesive ends at both ends, using a ligase, the DNA fragment group being obtained by the method according to claim 10.

13. A concatenated long-chain DNA amplification product that holds methylation information, obtained by carrying out amplification, using the concatenated long-chain DNA that holds methylation information according to claim 12 as a template.

14. A method for obtaining a DNA fragment group, comprising:
(1) digesting a DNA to be analyzed with a methylation-sensitive restriction enzyme whose recognition site includes methylated cytosine or cytosine to be methylated and which generates a cohesive end;
(2) ligating stem-loop adaptors which do not generate a recognition site of the methylation-sensitive restriction enzyme, to both ends of the DNA fragments obtained in step (1);
(3) digesting the DNA constructs obtained in step (2) with a methylation-insensitive restriction enzyme which recognizes the same recognition site as the methylation-sensitive restriction enzyme and which generates a cohesive end;
(4) ligating nuclease-resistant labeled adaptors whose 5′-end shows nuclease resistance and which generate the recognition site of the methylation-insensitive restriction enzyme, to each cohesive end of the DNA fragments obtained in step (3); and
(5) treating the DNA constructs obtained in step (4) with a single-strand-specific endonuclease followed by a combination of double-strand-specific and single-strand-specific endonucleases, to completely digest only DNA fragments to which the stem-loop adaptors are ligated, and to obtain a DNA fragment group consisting of DNA fragments to which the nuclease-resistant labeled adaptors are ligated at both ends thereof.

15. The method for obtaining the DNA fragment group, comprising:
(1) digesting the DNA fragment group obtained by the method according to claim 14 with the methylation-insensitive restriction enzyme described in claim 14; and
(2) removing the nuclease-resistant labeled adaptors from the digested product obtained in step (1) using a binding partner specific to the label, to obtain a DNA fragment group consisting of DNA fragments in which methylated cytosines are included in both cohesive ends at both ends.

16. A concatenated long-chain DNA that holds methylation information, obtained by multiple-ligating the DNA fragment group consisting of DNA fragments in which methylated cytosines are included in both cohesive ends at both ends, using a ligase, said DNA fragment group being obtained by the method according to claim 15.

17. A concatenated long-chain DNA amplification product that holds methylation information, obtained by carrying out amplification, using the concatenated long-chain DNA that holds methylation information according to claim 16 as a template.

18. A method for a DNA fragment group, comprising:
(1) digesting a DNA to be analyzed with a methylation-sensitive restriction enzyme whose recognition site includes methylated cytosine or cytosine to be methylated and which generates a cohesive end;
(2) ligating nuclease-resistant labeled adaptors whose 5′-end shows nuclease resistance and which have a restriction enzyme recognition site consisting of 8 nucleotides or more and do not generate a recognition site of the methylation-sensitive restriction enzyme, to both ends of the DNA fragments obtained in step (1);
(3) digesting the DNA constructs obtained in step (2) with a methylation-insensitive restriction enzyme which recognizes the same recognition site as the methylation-sensitive restriction enzyme and which generates a cohesive end;
(4) ligating stem-loop adaptors to both ends of the DNA fragments obtained in step (3);
(5) treating the DNA constructs obtained in step (4) with a single-strand-specific endonuclease followed by a combination of double-strand-specific and single-strand-specific endonucleases, to completely digest only DNA fragments to which the stem-loop adaptors are ligated, and to obtain a DNA fragment group consisting of DNA fragments to which the nuclease-resistant labeled adaptors are ligated at both ends thereof.

19. The method for the DNA fragment group, comprising:
(1) digesting the DNA fragment group obtained by the method according to claim 18, with a restriction enzyme which recognizes the restriction enzyme recognition site consisting of 8 nucleotides or more in the labeled adaptor described in claim 18; and
(2) removing the nuclease-resistant labeled adaptors from the digested product obtained in step (1) using a binding partner specific to the label, to obtain a DNA fragment group consisting of DNA fragments in which cytosines are included in both cohesive ends at both ends.

20. A concatenated long-chain DNA that holds methylation information, obtained by multiple-ligating the DNA fragment group consisting of DNA fragments in which cytosines are included in both cohesive ends at both ends, using a ligase, the DNA fragment group being obtained by the method according to claim 19.

21. A concatenated long-chain DNA amplification product that holds methylation information, obtained by carrying out amplification, using the concatenated long-chain DNA that holds methylation information according to claim 20 as a template.

22. The method for a DNA fragment group according to claim 10, wherein, after at least one digestion step of the restriction enzyme digestion steps or the nuclease digestion steps, a desired DNA fragment group is fractionated from the obtained mixture of DNA fragments.

23. A method for determining a methylation state in a DNA to be analyzed, comprising:
determining a nucleotide sequence of the concatenated long-chain DNA that holds methylation information according to claim 12, or the concatenated long-chain DNA amplification product that holds methylation information according to claim 13.
